(19) United States
(12) Patent Application Publication
(10) Pub. No.: US 2012/0029643 A1
(43) Pub. Date: Feb. 2, 2012

(54) RESTORATIVE DEVICE

(75) Inventor: Dror Robinson, Kfar Shmuel (IL)

(73) Assignee: CHI 2 GEL LTD, Ofakim (IL)

(21) Appl. No.: 13/258,526

(22) PCT Filed: Mar. 23, 2010
(86) PCT No.: PCT/IL10/00243

§ 371 Date: Sep. 22, 2011

Related U.S. Application Data
(60) Provisional application No. 61/162,367, filed on Mar. 23, 2009.

(51) Int. Cl. A61F 2/44

(52) U.S. Cl. 623/17.16

(57) ABSTRACT

The present invention provides a disc restoration system for relieving symptoms of a degenerative disc, comprising: a) a percutaneously insertable expandable element (88) adapted to be (i) inserted in between two adjacent vertebraes of the spinal cord; and, (ii) expanded to form a scaffold; said scaffold provides mechanical support to said two adjacent vertebraes so as to restore said degenerative disc to approximately the dimensions of a normal disc; b) an injectable filler having a first flowable state, and a second non-flowable set state; the filler is configured for being introduced, in said first flowable state, into a confined volume formed by the expandable element and the tissues, following the full expansion of said expandable element in said disc; and, following introduction of said filler, said filler is adapted to set into said second non-flowable state; such that following setting of said filler into said second state said disc is internally supported by said set filler.

### FIELD OF THE INVENTION

The present invention is directed towards means and methods of restoring damaged body organs (e.g., degenerative disc, torn tendon) or treatments of different diseases (e.g., cancer). More specifically the present invention relates alleviating symptoms due to degenerative disc. More specifically, the invention provides means and methods of restoring disc height as well as biomechanical properties and preventing the risk of eventual disc extrusion into the neural elements.

### BACKGROUND

The present invention is directed towards means and methods of restoring damaged body organs (e.g., degenerative disc, torn tendon) or treatments of different diseases (e.g., cancer).

The following description provides a mere example of use of the present invention in degenerative disc disease.

Degenerative disc disease refers to wear changes in the individual discs of the spine in any part of the spine. Spondylosis is another term for degenerative disc disease.

### Description of Degenerative Disc Disease (DDD).

DDD can affect any part of the spine, although common sites are the lumbar (lower back) and cervical (neck) spine; thoracic DDD is very uncommon.

Radiographic (x-ray) findings of DDD are a narrower disc space and some osteophyte (bony outgrowth of spur) formation. As people age, these changes tend to show up on the radiographs of most men and women. However, the first imaging modality to detect changes of DDD is MRI (magnetic resonance imaging), even before plain radiographs. Loss of water content (hydration) in the invertebral disc is an early finding, which is followed by narrowing of the disc space. People in the 20's and 30's may already have changes to their discs but no clinical symptoms. As the aging processes continues, the prevalence of DDD increases.

In the early phases of DDD, spontaneous or post-traumatic tears, degeneration, fibrosis, and collapse of the disc may lead to failure of mechanical function. This is associated with low back pain and possible leg pain if there is nerve root impingement (radiculopathy).

As DDD progresses, there is ligamentous buckling and osteophyte development which can cause narrowing of the space for the spinal cord and nerve roots. Lumbar spinal stenosis is the narrowing of the neural canal and foramina to an extent that results in confession of the lumbosacral nerve roots or cauda equina. Acquired lumbar stenosis is caused primarily by degenerative disease of the spine. However, a congenitally narrow or small spinal canal is a common finding; when present, it requires less disc degeneration, smaller disc herniation, or osteophytes to cause symptoms.

Furthermore, as s DDD progresses there would be an abnormal segmental motion with segmental instability.

### Causes and Risk Factors of Degenerative Disc Disease:

Degenerative disc disease can result from trauma (either acute or chronic/repetitive), infection, or the natural processes of aging. It can euphemistically be referred to as the “grey hairs of the spine”. The hallmark of DDD is dessication of the nucleus leading to its becoming smaller and thinner. A thin nucleus leads to bulging of the annulus fibers and pressure on nerves. The bulging fibers are mechanically disadvantaged and tend to undergo fraying and breakage. Thus nucleus herniation becomes possible.

### Symptoms of Degenerative Disc Disease:

The process of degeneration of the spine may lead to local pain, stiffness, and restricted activity. If there is disc herniation or rupture, one may also have leg/groin/knee pain dependent upon which nerve root is affected.

### Treatment of Degenerative Disc Disease:

In general the treatment of disc degeneration includes both (i) elevation of the disc height (which will releases pressure from the nerves and will restores facet orientation and prevents osteoarthritic pain); and (ii) restoring the mechanical properties (which will reduces abnormal motion and pain derived from end plate irritation).

Primary management for DDD is non-operative and includes nonsteroidal anti-inflammatory medications (NSAIDs) and exercise programs to strengthen abdominal and spinal musculature, improve aerobic fitness, and reduce lumbar lordosis (swayback).

Surgical intervention is an option when nonoperative medical management fails to adequately relieve the intolerable pain during activities of daily living which is individual-specific. It should also be considered in patients with initial signs and symptoms of progressive neurologic deterioration, specifically numbness or muscle weakness.

DDD which has failed non-operative management is classically treated by surgical spinal fusion. However, advances in disc replacement technologies have made disc replacement a viable option for many individuals. Early investigations have demonstrated lumbar disc replacements have had outcomes equivalent to spine fusion. Not all individuals with DDD are good candidates for disc replacement surgery. Concomitant spinal deformity (scoliosis, kyphosis), history of spinal infection, posterior spinal arthritis, and multilevel disc disease are relative or absolute contraindications.

For those individuals who are not candidates for lumbar disc replacement, lumbar spine fusions have had good short-term and long-term outcomes. Fusion involves creating a solid bony connection between two or more vertebrae anteriorly, posteriorly, or both.

In a spine fusion procedure, the surgeon joins two or more adjacent vertebrae. Bone taken from other parts of the body, usually the pelvis just above the hip joint, is placed across the vertebrae. Plugs of bone shaped like hockey pucks or cages made of metal or plastic are used between the vertebrae anteriorly. Posteriorly the bone is ground up into small pieces and laid down over the spine. The vertebrae and bone graft grow together as healing progresses, eventually forming a single unit without motion across them.

If the spine is in overall good position, spinal implants may not be necessary. So while not all spinal fusions require implants, many patients whose spines are weakened by injury or disease or whose deformities must be corrected are treated with internal fixation or spinal implants. If the spine needs to be placed and maintained in a new position, spinal implants will typically be necessary. The implants can include rods, screws, and hooks to fixate and stabilize the spine. Various types of implants are used depending on the problem that required the fusion, the patient's age, and the surgeon's judgment. These implants are usually left implanted indefinitely to minimize the possible loss of spinal alignment. The development of a spine fusion may take up to one year during which time physical activity may be limited and a spine brace may be recommended.

Fusion surgery is inherently more complicated, more painful, and riskier than procedures such as discectomy and laminectomy. There is no consensus in the medical community as to the appropriate indications for fusion surgery.

Nucleus pulposus is the jelly-like substance in the middle of the spinal disc. It is the remnant of the notochord. It functions to distribute hydraulic pressure in all directions within each disc under compressive loads. The nucleus pulposus consists of chondrocytes, collagen fibrils, and proteoglycan aggrecans that have hyaluronic long chains which attract water. Attached to each hyaluronic chain are side chains of chondroitin sulfate and keratan sulfate.

Herniated nucleus pulposus is a condition in which part or all of the soft, gelatinous central portion of an intervertebral disk is forced through a weakened part of the disk, resulting in back pain and nerve root irritation. Several solutions have been offered to alleviate this condition. U.S. Pat. No. 6,428,576 to Haldiman proposes a method of repairng a defect in an annulus fibrosus of an intervertebral disc. In this method a biocompatible flowable and curable material is introduced into the hollow space left by the defect. No solution apart from chemical crosslinking with the remaining annulus fibrosus is offered for confining the introduced material in the hollow space to prevent leakage. U.S. Pat. No. 6,140,452 to Felt et al provides for in situ tissue repair using polyurethane compositions. Such compositions are foreign to the body and have potentially unfavourable sequelae. U.S. Pat. No. 5,645,597 to Krapiva requires the removal of the natural disc, and insertion of a sac filled with a gel. The sac remains in situ. U.S. Pat. No. 7,258,700 to Lambrecht requires implantation of a metallic annulus replacement device to prevent leakage of the gel. Other prior art solutions also offer permanently implanted devices, which tend to have long term deleterious effects to the patient, including the development of wear debris with time, and implant displacement.

The US patent application of Donovan 2008/0249604 ('604) taught devices and methods for performing a procedure within a spine. A collapsed balloon is inserted into a patient's body and then, inflated (Prior Art FIG. 6a of the present application). The targeted cavity is provided with a filler through the opening at the distal end of the device. Simultaneously with the delivery of the filler material, withdrawal at least a portion of the medium from the expandable member is performed (Prior art FIG. 6 b,c,d of the present application). This procedure is fraught with difficulties for the surgeon, who has to judge very carefully the simultaneous delivery of filler and deflation of the balloon, in a sensitive and confined area of the body which leaves little room for error. Furthermore, due to the special configuration of the balloon of the present invention provides (i) mechanical support is provided to the disc and restores normal disc height; (ii) the filler can be delivered while the balloon is fully expanded; and, (iii) prevents leakage of the filler into the neural elements.

It is therefore a long and unmet need to provide means and methods of disc restoration which are efficacious over time, and as conservative as possible.

### SUMMARY OF THE INVENTION

It is one aspect of the invention to provide a disc restoration system for relieving symptoms of a degenerative disc, wherein said system comprising;
a) a percutaneously insertable expandable element adapted to be inserted into a degenerative disc of the spinal cord and expanded in said disc to form a scaffold; said scaffold provides mechanical support to said disc so as to restore said disc to approximately the dimensions of a normal disc; said expandable element is characterized by a predetermined 3D shape, such that upon expansion of said element, a confined volume within the same is created;
b) an injectable filler having a first flowable state, and a second non-flowable set state;
said filler is configured for being introduced, in said first flowable state, into said confined volume of said scaffold following the full expansion of said expandable element in said disc; and, following introduction of said filler into said confined volume of said scaffold, said filler is adapted to set into said second non-flowable state; such that following setting of said filler into said second state so as to internally support said disc by said set filler.

It is another aspect of the invention to provide the above mentioned disc restoration system, wherein said filler is selected from a group consisting of chitosan Fibrin, Hyaluronate, Hydroxyethylcellulose, Biosyntech glycerol phosphate/chitosan or any combination thereof.

It is another aspect of the invention to provide the above mentioned disc restoration system, wherein said expandable element is injected into the rotator cuff tears in the shoulder so as to limit the movement of said filler in said first flowable state until said filler is set into said second state.

It is another aspect of the invention to provide the above mentioned disc restoration system, wherein said scaffold includes scaffolding means selected from the group consisting of deployable arms, bulges, struts, swellings, irregular shapes and regular shapes or any combination thereof.

It is another aspect of the invention to provide the above mentioned disc restoration system, wherein said expandable element is configured for being removed from said disc within about 1-60 minutes after insertion of said expandable element, thereby leaving said chitosan filler in said set second state supporting said disc in situ.

It is another aspect of the invention to provide the above mentioned disc restoration system, wherein said expandable element is inflatable by a fluid via a lumen.

It is another aspect of the invention to provide the above mentioned disc restoration system, wherein said scaffold is balloon-like.

It is another aspect of the invention to provide the above mentioned disc restoration system, wherein said balloon-like scaffold is provided with means of insertion in an uninflated state between two adjacent vertebraes, further wherein said balloon-like scaffold is inflatable in situ so as to create space between said two adjacent vertebrae so as to provide mechanical support for the same.

It is another aspect of the invention to provide the above mentioned disc restoration system, wherein said balloon-like scaffold is provided with means of filling said space between said vertebrae with bone growth encouraging material.

It is another aspect of the invention to provide the above mentioned disc restoration system, wherein said balloon-like scaffold is provided with means of insertion intrapedicularly into spaces within a vertebral fracture further wherein said balloon-like scaffold is inflatable in situ so as to create space within said vertebral fracture.

It is another aspect of the invention to provide the above mentioned disc restoration system, wherein said balloon-like scaffold is provided with means of filling said space within said vertebral fracture with bone growth encouraging material.

It is another aspect of the invention to provide the above mentioned disc restoration system, wherein said scaffold has interstices that are narrower at a proximal site of injection of said filler into the interior of said raised disc than at sites distal to said site of injection, so as to facilitate gelation and setting of said filler in a proximal to distal direction, such that leakage of said filler is prevented.

It is another aspect of the invention to provide the above mentioned disc restoration system, wherein said expandable element additionally comprises a plurality of predetermined orifices from which said filler is introduced into said confined volume of said expandable element

It is another aspect of the invention to provide the above mentioned disc restoration system, wherein said scaffold has a spiral-like configuration.

It is another aspect of the invention to provide the above mentioned disc restoration system, wherein said chitosan filler comprises any chitosan hybrid

It is another aspect of the invention to provide the above mentioned disc restoration system, wherein said scaffold has a serpentine-like configuration.

It is another aspect of the invention to provide the above mentioned disc restoration system, wherein said scaffold has a jack-like configuration.

It is another aspect of the invention to provide the above mentioned disc restoration system, wherein said expandable element comprises at least one rigid or semi rigid members.

It is another aspect of the invention to provide the above mentioned disc restoration system, wherein said scaffold is formed from shape memory alloys.

It is another aspect of the invention to provide the above mentioned disc restoration system, wherein said expandable element is provided with means for sustained drug delivery.

It is another aspect of the invention to provide the above mentioned disc restoration system, wherein said expandable element is provided with means for rapid drug delivery.

It is another aspect of the invention to provide the above mentioned disc restoration system, wherein said expandable element is provided with a removable posterior wall supporting means.

It is an aspect of the invention to provide the above mentioned disc restoration system, wherein said filler additionally comprises endogenous or non-endogenous disc-cells

It is another aspect of the invention to disclose a method for relieving symptoms of a degenerative disc, comprising steps of;
a. obtaining a disc restoration system, said system comprising;
   (i) a percutaneously insertable expandable element adapted to be inserted into a degenerative disc of the spinal cord and expanded in said disc to form a scaffold; said scaffold provides mechanical support to said disc so as to restore said disc to approximately the dimensions of a normal disc; said expandable element is characterized by a predetermined 3D shape, such that upon expansion of said element, a confined volume within the same is created;
   (ii) an injectable filler having a first flowable state, and a second non-flowable set state;
b. inserting said expandable element into said degenerative disc;
c. fully expanding said element so as to restore said disc to approximately normal disc dimensions;
d. introducing said filler into said confined volume of said expandable element; thereby filling the internal spaces of said restored disc with said filler;
e. waiting for said filler to gelate and set to said second state; and,
f. removing said expandable element.

It is another aspect of the invention to disclose the above mentioned method, additionally comprising step of providing said expandable element with scaffolding means selected from a group consisting of deployable arms, bulges, struts, swellings, irregular shapes and regular shapes or any combination thereof.

It is another aspect of the invention to disclose the above mentioned method, additionally comprising step of withdrawing said expandable element from the interior of said disc within about 1-60 minutes following insertion of said expandable element, thereby leaving said filler in said gelated second state supporting said disc in situ

It is another aspect of the invention to disclose the above mentioned method, wherein said step of fully expanding said element is performed by a fluid via a lumen.

It is another aspect of the invention to disclose the above mentioned method, additionally comprising step of selecting said filler from a group consisting of chitosan Fibrin, Hyaluronate, Hydroxyethylcellulose, Biosyntech glycerol phosphate/chitosan or any combination thereof

It is another aspect of the invention to disclose the above mentioned method, wherein said chitosan filler comprises any chitosan hybrid.

It is another aspect of the invention to disclose the above mentioned method, additionally comprising step of configuring saod scaffold to have a balloon-like structure.

It is another aspect of the invention to disclose the above mentioned method, additionally comprising step of injecting said expandable element into the rotator cuff tears in the shoulder; thereby limiting the movement of said filler in said first flowable state until said filler is set into said second state.

It is another aspect of the invention to disclose the above mentioned method, wherein said method further comprises steps of:
a. inserting said uninflated balloon-like scaffold between two adjacent vertebrae; and,
b. inflating said balloon-like scaffold in situ so as to create space between said vertebrae.

It is another aspect of the invention to disclose the above mentioned method, further comprising steps of filling said space between said vertebrae with bone growth encouraging material.

It is another aspect of the invention to disclose the above mentioned method wherein said method further comprises steps of:
a. inserting intrapedicularly said balloon-like scaffold into spaces within a vertebral fracture;
b. inflating said balloon-like scaffold e in situ so as to create space within said vertebral fracture;

It is another aspect of the invention to disclose the above mentioned method further comprising steps of filling said spaces within said vertebral fracture with bone growth encouraging material.

It is another aspect of the invention to disclose the above mentioned method, additionally comprising step of providing said scaffold with interstices that are narrower at the proximal site of injection of said chitosan filler into the interior of said raised disc than at sites distal to said site of injection, thereby facilitating gelation and setting of said chitosan filler in a proximal to distal direction, thereby preventing leakage of said filler.

It is another aspect of the invention to disclose the above mentioned method, additionally comprising step of configuring said scaffold in a configuration selected from a spiral-like configuration, a serpentine-like configuration or any combination thereof.

It is another aspect of the invention to disclose the above mentioned method, additionally comprising step of configuring said scaffold in a jack-like configuration.

It is another aspect of the invention to disclose the above mentioned method, additionally comprising step of providing said expandable element with at least one rigid or semi rigid members.

It is another aspect of the invention to disclose the above mentioned method, additionally comprising step of forming said scaffold from shape memory alloys.

It is a further aspect of the invention to disclose the above mentioned method, additionally comprising step of providing said expandable element with means for sustained drug delivery.

It is a further aspect of the invention to disclose the above mentioned method, additionally comprising step of providing said filler with endogenous or non-endogenous disc-cells

Moreover, it is another aspect of the invention to disclose the above mentioned method, additionally comprising step of providing said expandable element with means for rapid drug delivery.

Lastly is another aspect of the invention to disclose the above mentioned method, additionally comprising step of providing said expandable element with a removable posterior wall supporting means.

It is another object of the present invention to provide a restoration system; wherein said system comprising:
a) at least one percutaneously insertable expandable element adapted to be (i) inserted into a region of interest in the body; and, (ii) expanded in the same to form a scaffold;
said expandable element is characterized by a predetermined 3D shape, such that upon expansion of said element, a confined volume within the same is created;
b) an injectable filler having a first flowable state, and a second non-flowable set state;
said filler is configured for being introduced, in said first flowable state, into said confined volume of said scaffold following the full expansion of said two expandable elements in said region of interest so as to prevent any leakage of said filler out of said confined volume; and, following introduction of said filler into said confined volume of said scaffold, said filler is adapted to set into said second non-flowable state.

It is another object of the present invention to provide the restoration system as defined above, wherein said expandable element comprises at least two coaxial cylindrically shaped balloons.

It is another object of the present invention to provide the restoration system as defined above, wherein said region of interest in the body is suspicious to be cancerous; such that said cancer is confined within the inner space between two neighboring balloons; further wherein said filler is a chemotherapy agent released from the outer balloon towards said cancer, such that said restoration system is used for treating said cancer.

It is another object of the present invention to provide the restoration system as defined above, wherein said restoration system is used for treating cancer.

It is another object of the present invention to provide the restoration system as defined above, wherein said region of interest is selected from pancreas, Pancreas, liver and other soft tissue.

It is another object of the present invention to provide the restoration system as defined above, wherein said filler is selected from a group consisting of chitosan Fibrin, Hyaluronate, Hydroxyethylcellulose, Biosyntech glycerol phosphate/chitosan or any combination thereof.

It is another object of the present invention to provide the restoration system as defined above, wherein said expandable element is injected into the rotator cuff tears in the shoulder so as to limit the movement of said filler in said first flowable state until said filler is set into said second state.

It is another object of the present invention to provide the restoration system as defined above, wherein said scaffold includes scaffolding means selected from the group consisting of deployable arms, bulges, struts, swellings, irregular shapes and regular shapes or any combination thereof.

It is another object of the present invention to provide the restoration system as defined above, wherein said expandable element is configured for being removed from said disc within about 1-60 minutes after insertion of said expandable element, thereby leaving said filler in said set second state supporting said disc in situ.

It is another object of the present invention to provide the restoration system as defined above, wherein said expandable element is inflatable by a fluid via a lumen.

It is another object of the present invention to provide the restoration system as defined above, wherein said scaffold is balloon-like.

It is another object of the present invention to provide the restoration system as defined above, wherein said balloon-like scaffold is provided with means of insertion in an uninflated state between two adjacent vertebraes, further wherein said balloon-like scaffold is inflatable in situ so as to create space between said two adjacent vertebrae so as to provide mechanical support for the same.

It is another object of the present invention to provide the restoration system as defined above, wherein said balloon-like scaffold is provided with means of filling said space between said vertebrae with bone growth encouraging material.

It is another object of the present invention to provide the restoration system as defined above, wherein said balloon-like scaffold is provided with means of insertion intrapedicularly into spaces within a vertebral fracture; further wherein said balloon-like scaffold is inflatable in situ so as to create space within said vertebral fracture.

It is another object of the present invention to provide the restoration system as defined above, wherein said balloon-like scaffold is provided with means of filling said space within said vertebral fracture with bone growth encouraging material.

It is another object of the present invention to provide the restoration system as defined above, wherein said scaffold has interstices that are narrower at a proximal site of injection of said filler into the interior of said raised disc than at sites distal to said site of injection, so as to facilitate gelation and setting of said filler in a proximal to distal direction, such that leakage of said filler is prevented.

It is another object of the present invention to provide the restoration system as defined above, wherein said expandable element additionally comprises a plurality of predetermined orifices from which said filler is introduced into said confined volume of said expandable element.

It is another object of the present invention to provide the restoration system as defined above, wherein said scaffold has a spiral-like configuration.

It is another object of the present invention to provide the restoration system as defined above, wherein said scaffold has a serpentine-like configuration.

It is another object of the present invention to provide the restoration system as defined above, wherein said scaffold has a jack-like configuration.

It is another object of the present invention to provide the restoration system as defined above, wherein said expandable element comprises at least one rigid or semi rigid member.

It is another object of the present invention to provide the restoration system as defined above, wherein said scaffold is formed from shape memory alloys.

It is another object of the present invention to provide the restoration system as defined above, wherein said expandable element is provided with means for sustained drug delivery.

It is another object of the present invention to provide the restoration system as defined above, wherein said filler additionally comprises nano-particles.

It is another object of the present invention to provide the restoration system as defined above, wherein said expandable element is provided with means for rapid drug delivery.

It is another object of the present invention to provide the restoration system as defined above, wherein said expandable element is provided with a removable posterior wall supporting means.

It is another object of the present invention to provide the restoration system as defined above, wherein said filler additionally comprises endogenous or non-endogenous disc-cells.

It is another object of the present invention to provide the restoration system as defined above, wherein said at least two elements are adapted to apply pressure on an organ enclosed within said confined volume.

It is another object of the present invention to provide the restoration system as defined above, wherein said organ is selected from a group consisting of tendon, Pancreas, liver and other soft tissue.

It is another object of the present invention to provide a method for treating a body organ, comprising steps of;
a. obtaining a restoration system, said system comprising;
   i. at least one percutaneously insertable expandable element adapted to be (i) inserted into a region of interest in the body; and, (ii) expanded in the same to form a scaffold; said expandable element is characterized by a predetermined 3D shape, such that upon expansion of said element, a confined volume within the same is created;
   ii. an injectable filler having a first flowable state, and a second non-flowable set state;
b. inserting said two expandable element into said region of interest;
c. expanding said inner element so as to circumscribe said body organ;
d. expanding said outer element thereby creating said confined volume;
e. introducing said filler into said confined volume thereby treating said cancer; and,
f. removing said expandable element.

It is another object of the present invention to provide the method as defined above, additionally comprising step of providing said expandable element with at least two coaxial cylindrically shaped balloons.

It is another object of the present invention to provide the method as defined above, wherein said region of interest in the body is suspicious to be cancerous; such that said cancer is confined within the inner space between two neighboring balloons; further wherein said filler is a chemotherapy agent released from the outer balloon towards said cancer, such that said restoration system is used for treating said cancer.

It is another object of the present invention to provide the method as defined above, wherein said organ is selected from a group consisting of tendon.

It is another object of the present invention to provide the method as defined above, additionally comprising step of providing said expandable element with scaffolding means selected from a group consisting of deployable arms, bulges, struts, swellings, irregular shapes and regular shapes or any combination thereof.

It is another object of the present invention to provide the method as defined above, additionally comprising step of withdrawing said expandable element from the interior of said disc within about 1-60 minutes following insertion of said expandable element, thereby leaving said filler in said gelated second state supporting said disc in situ.

It is another object of the present invention to provide the method as defined above, wherein said step of fully expanding said element is performed by a fluid via a lumen.

It is another object of the present invention to provide the method as defined above, additionally comprising step of selecting said filler from a group consisting of chitosan Fibrin, Hyaluronate, Hydroxyethylcellulose, Biosyntech glycerol phosphate/chitosan or any combination thereof.

It is another object of the present invention to provide the method as defined above, additionally comprising step of configuring saod scaffold to have a balloon-like structure.

It is another object of the present invention to provide the method as defined above, additionally comprising step of injecting said expandable element into the rotator cuff tears in the shoulder; thereby limiting the movement of said filler in said first flowable state until said filler is set into said second state.

It is another object of the present invention to provide the method as defined above, wherein said method further comprises steps of:
a. inserting said uninflated balloon-like scaffold between two adjacent vertebrae; and,
b. inflating said balloon-like scaffold in situ so as to create space between said vertebrae.

It is another object of the present invention to provide the method as defined above, further comprising steps of filling said space between said vertebrae with bone growth encouraging material.

It is another object of the present invention to provide the method as defined above, wherein said method further comprises steps of:
a. inserting intrapedicularly said balloon-like scaffold into spaces within a vertebral fracture;
b. inflating said balloon-like scaffold in situ so as to create space within said vertebral fracture.

It is another object of the present invention to provide the method as defined above, further comprising step of filling said spaces within said vertebral fracture with bone growth encouraging material.

It is another object of the present invention to provide the method as defined above, additionally comprising step of providing said scaffold with interstices that are narrower at the proximal site of injection of said chitosan filler into the interior of said raised disc than at sites distal to said site of injection, thereby facilitating gelation and setting of said chitosan filler in a proximal to distal direction, thereby preventing leakage of said filler.

It is another object of the present invention to provide the method as defined above, additionally comprising step of configuring said scaffold in a configuration selected from a spiral-like configuration, a serpentine-like configuration or any combination thereof.

It is another object of the present invention to provide the method as defined above, additionally comprising step of configuring said scaffold in a jack-like configuration.

It is another object of the present invention to provide the method as defined above, additionally comprising step of providing said expandable element with at least one rigid or semi rigid members.

It is another object of the present invention to provide the method as defined above, additionally comprising step of forming said scaffold from shape memory alloys.

It is another object of the present invention to provide the method as defined above, additionally comprising step of providing said expandable element with means for sustained drug delivery.

It is another object of the present invention to provide the method as defined above, additionally comprising step of providing said expandable element with means for rapid drug delivery.

It is still an object of the present invention to provide the method as defined above, additionally comprising step of providing said expandable element with a removable posterior wall supporting means.

It is lastly an object of the present invention to provide the method as defined above, additionally comprising step of providing said filler with endogenous or non-endogenous disc-cells.

### BRIEF DESCRIPTION OF FIGURES

In order to understand the invention and to see how it may be implemented in practice, a few preferred embodiments will now be described, by way of non-limiting example only, with reference to the accompanying drawing, in which;

FIG. 1 is a cross sectional schematic representation of a vertebra.

FIG. 2 is a cross sectional schematic representation of an embodiment of the invention.

FIGS. 3-13 are schematic representations of an embodiment of the invention.

FIG. 14 is a schematic representation of a prior art embodiment.

FIGS. 15 -17 illustrates different examples of the expandable element.

FIGS. 18-25 illustrates different uses to the device of the present invention.

FIG. 26 illustrates restoration of the disc's mechanical properties by the implantation of the filler.

FIG. 27 illustrates the disc behavior under compression of a normal disc, a degenerated disc, a no cross linking—no hyaluronate, a no cross linking with hyaluronate, a 40 minutes cross linking with gennipin, a 1 minute cross linking with gennipin).

FIG. 28 again illustrates the restoration of mechanical properties while using the device of the present invention.

FIG. 29 illustrates the increase (%) in neural foramen size.

FIG. 30 illustrates the disc height increase when a balloon and filler are used and when the filler alone is used in different models.

FIG. 31 illustrates the palpatory size of the subcutaneous gel/filler used in the present invention.

FIG. 32 illustrates changes in disc behavior under compression of a normal disc and of a degenerated disc.

FIG. 33 illustrates the ability of the repair disc to shock absorb.

FIG. 34 illustrates the amount of translation in a normal disc, a degenerated disc and in a repaired disc after 20 Kg force was applied.

FIG. 35 illustrates the % Wet degeneration after 5 days incubation at 37 degrees in 0.2% lysozyme comparing different % of chitosan and Hyaluronate (HA)s the filler.

FIG. 36 illustrates the disc height as a function of time after disc injury induction of an experiment performed on 8 rabbits.

FIG. 37 illustrates the Osteophytes' score (0-4 grading) in which 0 means—no osteophytes; 1 means minimal osteophyte; 2 meansosteophyte larger than half disc height; 3 almost touching osteophytes; and, 4 ankylosis.

### DETAILED DESCRIPTION OF THE INVENTION

In the following description, various aspects of the invention will be described. For the purposes of explanation, specific configurations and details are set forth in order to provide a thorough understanding of the invention. However, it will be also apparent to one skilled in the art that the invention may be practiced without specific details presented herein. Furthermore, well-known features may be omitted or simplified in order not to obscure the invention.

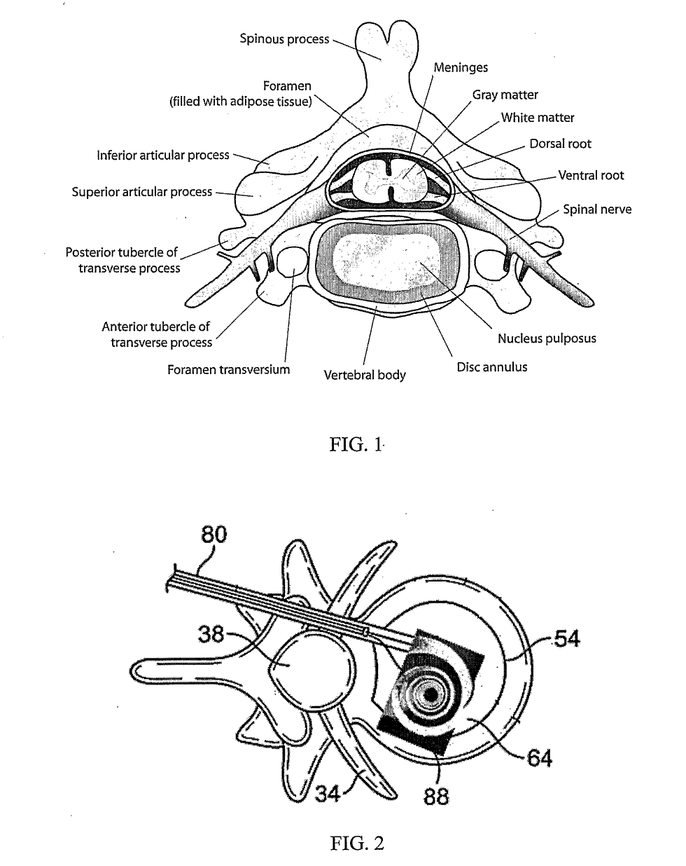

FIG. 1 represents the cross section of a vertebra.

FIG. 2 is an example of an embodiment of the invention in which 38 is the gray matter; 80 is an introducer; 88 is the spiral portion of the expandable element, namely the balloon; 64 is the nucleus pulposus; 54 is the annulus fibrosus; 34 is the spinal nerve.

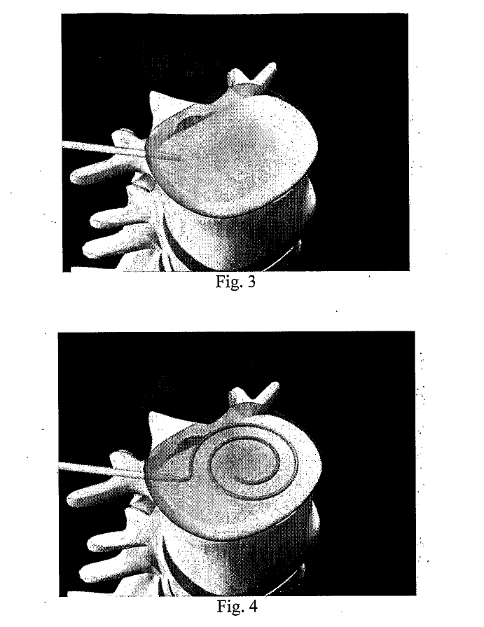

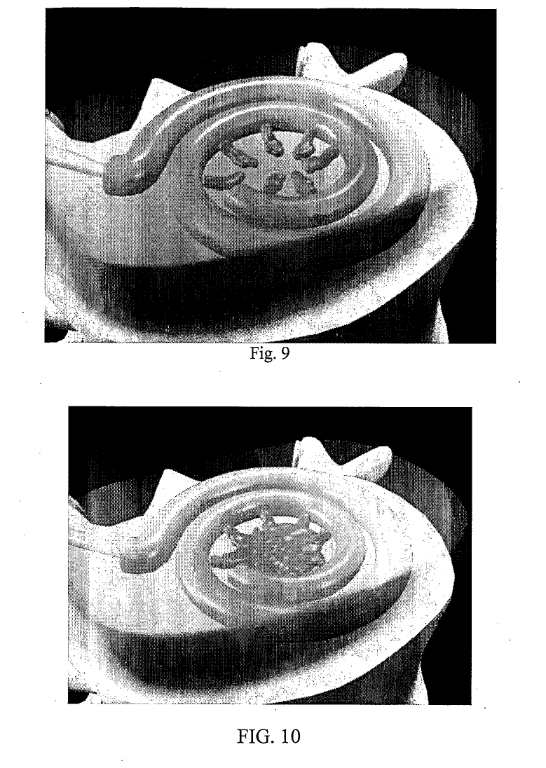

FIGS. 3-13 are schematic representations of an embodiment of the invention. The percutaneously insertable expandable element is being inserted into a degenerative disc of the spinal cord and is expanding in the disc to form a scaffold, so as to restore said disc to approximately the dimensions of a normal disc.

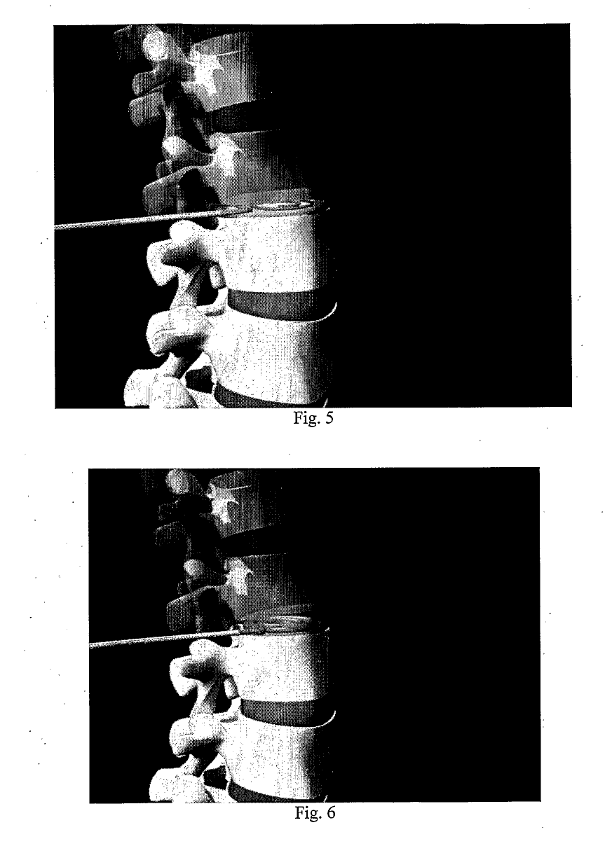

FIGS. 3 and 4 demonstrate the introduction of the expandable element into a degenerative disc of the spinal cord. In this example the expandable element is a balloon.

In FIG. 5 the expandable element is fully introduced into the degenerative disc and the same is being to inflate.

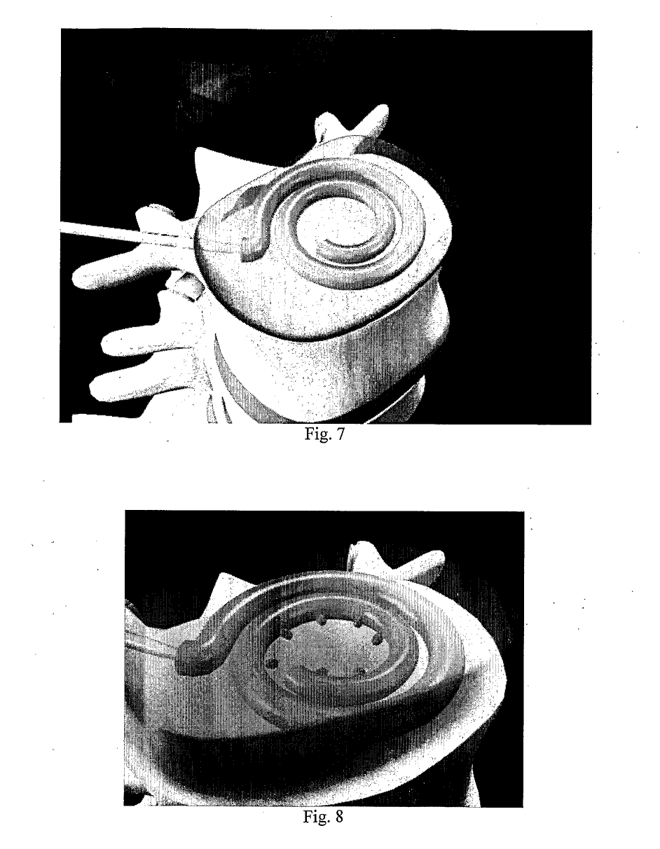

FIGS. 6 and 7 illustrate the expandable element in its fully expanded configuration to form a scaffold. In said configuration the expandable element (i.e., the scaffold) provides mechanical support to the degenerative disc and prevents any collapse of two adjacent vertebrae.

Furthermore, once the expandable element is expanded—a confined volume is created into which the filler will be introduced to.

Now, once the expandable element is fully expanded, the filler is introduced.

It should be emphasized that the filler is introduced, in its first flowable state, into the scaffold following expansion of said expandable element in said disc. In this example, the filler flows into the disc cavity through predetermined orifices in the expandable element, which, in this example as mentioned above, is balloon like.

FIGS. 8-11 illustrates the introduction of said filler through said predetermined orifices in the expandable element.

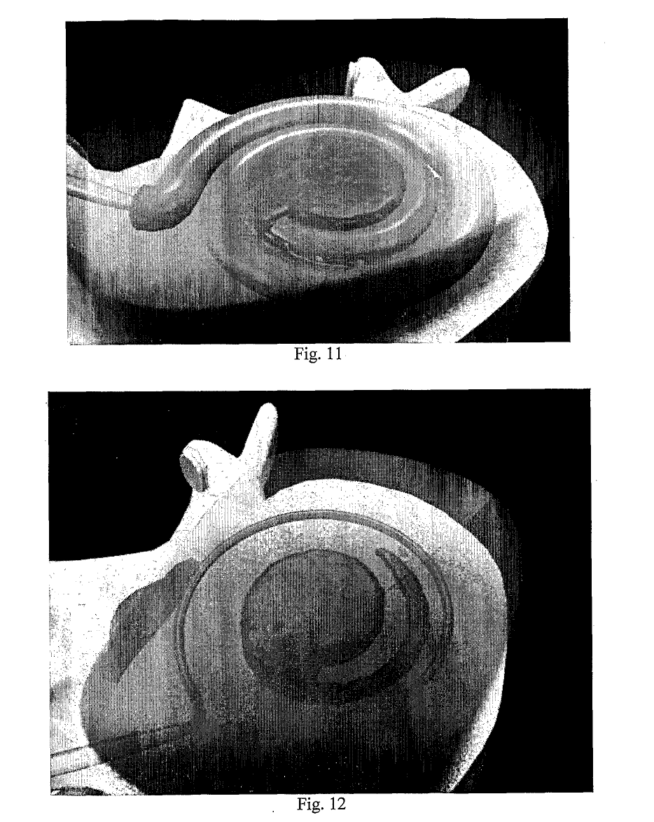

As can be seen in FIG. 11, the expandable element, once fully expanded, creates a confined volume within the same, into which the filler is introduced.

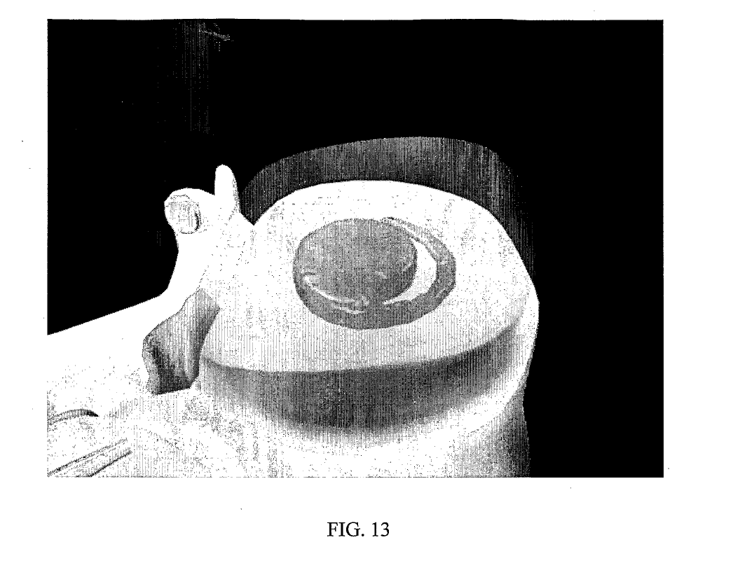

FIG. 12 is a schematic illustration of an embodiment of the invention following introduction of the filler into the scaffold. The filler has set into the second non-flowable state. Following setting of the filler into the second state, the scaffold has been removed from the disc (see FIGS. 12-13), resulting in the disc being internally supported and raised by the set filler.

Now the filler infiltrates the crevices and fills the tissue from within and not as one bulk.

It should be emphasized that the use of the system of the present invention enables the following:
(i) restoration of disc height;
(ii) increase of neural formation size;
(iii) decreases pressure on the neural elements, while preserving motion of the functional motion segment.

The gel/filler used can be selected from Fibrin, Hyaluronate, Hydroxyethylcellulose, Biosyntech glycerol phosphate/chitosan or any combination thereof.

The main features of the device are as follows:
(i) the device is percutaneously implantable;
(ii) The device restores disc height;
(iii) The gel is injectable percutaneously;
(iv) The balloon-gel procedure restores mechanical properties of the degenerated disc;
(v) The gel/filler coagulates in situ;
(vi) The gel/filler does not leak provided a balloon elevation device is used
(vii) The gel/filler does not dislodge during at least 3 months follow-up period;
(viii) Osteophyte production is halted after disc injury that should cause disc degeneration;
(ix) Crevice formation within damaged discs is halted;
(x) The gel/filler relieves osteoarthritic pain;
(xi) The gellfiller undergoes slow biodegradation that is completed within 4 months;
(xii) Histologicallly the disc degenerative process is halted and new tissue formation is seen;
(xiii) The gel/filler does not evoke an inflammatory response
(xiv) Biosyntech gel/filler inferiority due to cell toxicity of their gel/filler; and,
(xv) Superiority to hyaluronate in stress conditions that are typical of degenerated discs.

According to a preferred embodiment of the present invention, the gel/filler is thermal and pH responsive and gelates upon tissue contact. Thus, it does not leak out of a perforated annulus provided a balloon off-loads the disc.

As it is widely known, the human degenerated disc is typified by annular cracks; thus an eminent danger of injections into it is the leakage of material to the neural elements.

According to another embodiment of the present invention the gel/filler of the present invention is hyperelastic, thus when extended to about 20 percent strain, it does not absorb energy. Hence, it indicates superior fatigue performance.

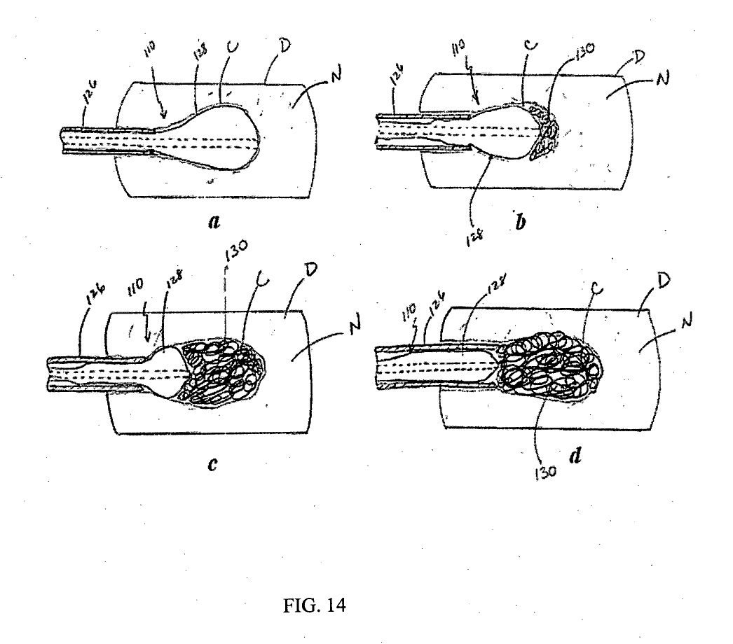

FIG. 14 is a schematic illustration of the prior art of Donovan US2008/0249604. A collapsed balloon is inserted into a patient's body and then, inflated (Prior Art FIG. 14a of the present application). The targeted cavity is provided with a filler through the opening at the distal end of the device. Simultaneously with the delivery of the filler material, withdrawal at least a portion of the medium from the expandable member is performed (Prior art FIG. 14b,c,d of the present application). The expandable member has to be at least partially deflated so as to enable the insertion of the filler.

Such device does not and can not provide constant mechanical support to the discs (i.e., to two adjacent vertebrae) all through the procedure since the introduction of the filler to the degenerate disc is accompanied by at partially deflating the expandable member. Such deflation will most probably result in the collapse of two adjacent vertebrae.

It should be emphasized that the device of the present invention enables/provides mechanical support to the discs due to the special 3D shape of the same which, upon expansion, creates a confined volume into which the filler is injected.

The expandable element of Donovan (US application US2008/0249604) does not produce a confined volume; therefore, in order to introduce the filler, the expandable element has to be at least partially deflated (so as to ‘clear out’ space to the filler).

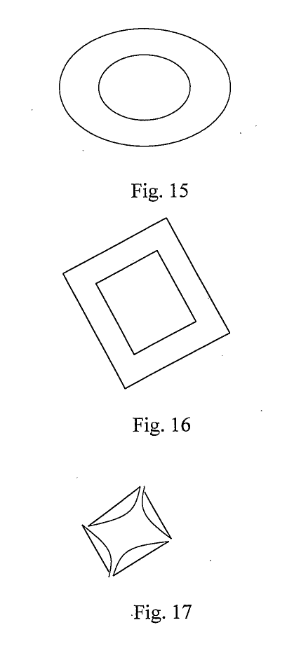

Reference is now made to FIGS. 15-17 which illustrates several embodiments of the different possible expandable elements.

FIGS. 15 and 16 illustrates continues round and square shape of the expandable element respectfully.

FIG. 17—in which the expandable element comprises several joints which provides the same with flexibility.

It is acknowledged herein that in some embodiments of the invention the term “chitosan” or “chitosan filler” may also refer to chitosan hybrids which can be used as fillers. In some embodiments of the invention the aforementioned chitosan filler used for filling the disc is a chitosan-GAG crosslinked hybrid.

Chitosan is a naturally occurring polysugar similar in structure to ECM constituents (GAGs).

Common chitosan is known to be positively charged and to precipitate at pH levels above 6.5. Usually mixing common chitosan with any negatively charged GAGs (Glycos-amino-glycans) results in fast precipitation (e.g. chitosan with HA, with chondroitin-sulphate)

A homogenous hybrid of common chitosan with negatively charged polymers is used in some embodiments of the invention and requires pH of 7 and above. This is possible through either of the following:
1. Mixing chitosan with polyols such as bGlycerolphosphate (Biosyntech Ltd).
2. Mixing chitosan with soluble-chitosan (i.e homogenously 50% acetylated chitosan—Chi2Gel Ltd) The datasheet and disclosure of LFG is incorporated in it's entirety (see APPENDIX I)
3. Using chemically modified soluble chitosan such as Kytogenic's NOCC technology (see Fertility and Sterility, Vol. 80, No.3 Sep. 2003, APPENDIX II, the datasheet and disclosure of Kitogenics, which is incoporated herein in its entirety).

It is further acknowledged herein that in some embodiments of the invention the aforementioned chitosan filler may be any polysugar, protein, GAGs, PLA (Polylactic acid), PGA(Polyglycolic acid), PLGA(poly lactic-co-glycolic acid) or a hybrid/mixture of such that can be made to gel either physically or using a cross-linking agent such as DVS (divynilsulfon), Geninip, GA (Glutaraldehyde).

See International patent application PCT/IL2007/001530 of Ben-shalom, Nevo, Patchornik and Robinson

A core principle of the invention is the means and method provided herein for preventing a degenerate or diseased disc extruding into the neural elements, and embodiments of the invention have been directed to this purpose.

A core principle of the invention is to provide a disc restoration device for relieving symptoms of a degenerative disc. The device comprises
a. a percutaneously insertable expandable element for inserting into said disc and expanding same, thereby forming a supporting temporary scaffold with interstices, and
b. a polymer possibly chitosan filler for filling internal spaces of said expanded disc.

The aforementioned chitosan filler is insertable into said disc and flowable around said scaffold in a first state, and gelatable, settable and non flowable in a second state. Moreover, the expandable element is adapted for raising said disc to about normal disc dimensions such that said chitosan filler in said first state can be injected into the interior of said raised disc and flow into said scaffolding interstices. The filler is adapted to gelate and set in said second state. The expandable element is removable after gelation and setting of said chitosan filler in said second state such that said disc is supported internally by said gelated, set and non flowing chitosan filler. This novel invention allows the surgeon more time to work within the very confined space, and is easier for the surgeon, since it separates the removal of the expandable element from injection of the chitosan filler. This is in complete contrast to the prior art of Donovan which teaches simultaneous delivery of the filler material and withdrawal of the expandable member (Prior art FIG. 6 b,c,d of the present application).

Exemplary embodiments of the invention are provided wherein the insertable expandable element is a balloon, which can be spiral or any other convenient shape which is adapted and designed to prevent leakage of fluid toward the posterior elements. The balloon is inflatable, and is able to raise the disc and support the disc, leaving intersticies in the nucleus pulpulosus. The intersticies may be labrinthine in nature, irregular or regular. The interstices themselves may be filled by injection with a chitosan filler of special gelatable, hardening, weight bearing, pressure bearing and setting properties such that within less than about 60 minutes the insertable expandable balloon may be removed, leaving the disc supported by the chitosan filler which has filled the labrinthine spaces, hardened and is able to support the treated disc. FIG. 2 is an example of an embodiment of the invention: within the figure, 88 denotes the insertable expandable element, 80 represents the insertion and removal device with 82 representing a device for inflating the expandable element and, in some embodiments, for injecting intersticies with gel.

In other embodiments of the invention the expandable element is a jack-like mechanical device which can likewise be removed, after the chitosan filler has been injected into the intersticies formed by the jack like mechanical device in the nucleus pulpulosus. It is herein acknowledged that the aforementioned device may be provided with a lumen for injecting the aforementioned chitosan filler. It is further envisaged that the chitosan filler composition, in some embodiments, may require an additional crosslinking material to be injected and integrated with it in situ to enable setting, hardening, curing and gelation to occur within a short time. It is further acknowledged that some embodiments of the device are provided with a lumen for the injection of the aforementioned crosslinking material.

Embodiments of the invention are herein provided wherein the expandable element of the disc restoration device is provided with scaffolding means selected from the group consisting of deployable arms, bulges, struts, swellings, irregular shapes and regular shapes or any combination thereof.

Embodiments of the invention are herein provided wherein the aforementioned expandable member is adapted for withdrawal from interior of said disc within about 1 minutes to about 60 minutes of insertion of said expandable member, thereby leaving said chitosan filler in said gelated second state supporting said disc in situ.

Embodiments of the invention are herein provided wherein the said expandable member is inflatable by a fluid via a lumen.

Embodiments of the invention are herein provided wherein the aforementioned scaffolding means is adapted is adapted to form labrinthine like intersticies within the nucleus pulposus.

In another embodiment of the device the spiral or otherwise shaped balloon is inserted into the disc space and a material encouraging bone formation is injected resulting in fusion of the intervertebral disc to the adjacent vertebrae.

In another embodiment of the device the balloon is inserted intrapedicularly or through the endplate and bone forming substance is injected into the vertebrae to ecourage healing of vertebral fractures.

Embodiments of the invention are herein provided wherein the aforementioned scaffolding means is balloon-like

Embodiments of the disc restoration device are herein provided wherein the aforementioned wherein said interstices of said scaffolding are narrower at the proximal site of said injection of said chitosan filler into the interior of said raised disc than at sites distal to said site of injection, thereby facilitating said gelation and said setting of said chitosan filler in a proximal to distal direction, thereby preventing leakage of said chitosan filler.

Further embodiments of the disc restoration device are herein provided wherein the said scaffolding means is spiral-like.

Further embodiments of the aforementioned disc restoration device are provided with said scaffolding means which are serpentine-like.

Further embodiments of the invention are provided wherein said scaffolding means is jack-like.

Further embodiments of the invention are provided wherein said expandable member comprises rigid or semi rigid members

Further embodiments of the invention are provided wherein said scaffolding means comprises shape memory alloys.

Further embodiments of the invention are provided wherein said expandable member is provided with means for sustained drug delivery.

Further embodiments of the invention are provided wherein said expandable member is provided with means for rapid drug delivery

Further embodiments of the invention are provided wherein said device is provided with a removable posterior wall supporting means.

A method for relieving symptoms of a degenerative disc is herein disclosed comprising steps of
a. obtaining a disc restoration device, said device comprising
   i. a percutaneously insertable expandable element for inserting into said disc and expanding same, thereby forming a supporting scaffold with interstices,
   ii. a chitosan filler for filling internal spaces of said expanded disc
      wherein said chitosan filler is insertable into said disc and flowable around said scaffold in a first state, and gelatable, settable and non flowable in a second state, further wherein said expandable element is adapted for raising said disc to about normal disc dimensions such that said chitosan filler in said first state can be injected into the interior of said raised disc and flow into said scaffolding interstices thereto gelate and set in said second state, and said expandable element is removable after gelation and setting of said chitosan filler in said second state such that said disc is supported internally by said gelated, set and non flowing chitosan filler and
a) inserting said expandable element into said degenerative disc;
b) expanding said disc;
c) injecting into and then filling said internal spaces of said expanded disc with said chitosan filler;
d) waiting for said chitosan filler to gelate and set; and,
e) removing said expandable element.

The above mentioned method is disclosed wherein said expandable element is provided with scaffolding means selected from the group consisting of deployable arms, bulges, struts, swellings, irregular shapes and regular shapes or any combination thereof.

The above mentioned method is disclosed wherein said expandable member is adapted for withdrawal from interior of said disc within about 60 minutes to about 10 minutes of insertion of said expandable member, thereby leaving said chitosan filler in said gelated second state supporting said disc in situ.

The above mentioned method is disclosed wherein said expandable member is inflatable by a fluid via a lumen.

The above mentioned method is disclosed wherein said scaffolding means is balloon-like.

The above mentioned method is disclosed wherein said interstices of said scaffolding are narrower at the proximal site of said injection of said chitosan filler into the interior of said raised disc than at sites distal to said site of injection, thereby facilitating said gelation and said setting of said chitosan filler in a proximal to distal direction, thereby preventing leakage of said chitosan filler.

The above mentioned method is disclosed wherein said scaffolding means is spiral-like.

The above mentioned method is disclosed wherein said scaffolding means is serpentine-like.

The above mentioned method is disclosed wherein said scaffolding means is jack-like.

The above mentioned method is disclosed wherein said expandable member comprises rigid or semi rigid members.

The above mentioned method is disclosed wherein said scaffolding means comprises shape memory alloys.

The above mentioned method is disclosed wherein said expandable member is provided with means for sustained drug delivery.

The above mentioned method is disclosed wherein said expandable member is provided with means for rapid drug delivery.

The above mentioned method is disclosed wherein said device is provided with a removable posterior wall supporting means.

According to another embodiment of the present invention, the balloon provided is adapted to create a volume in the tissue. Such an embodiment can be used to treat cancer, fuse tattered tendon's two end.

As described, according to another embodiment of the present invention, the device of the present invention is used in cancer treatment.

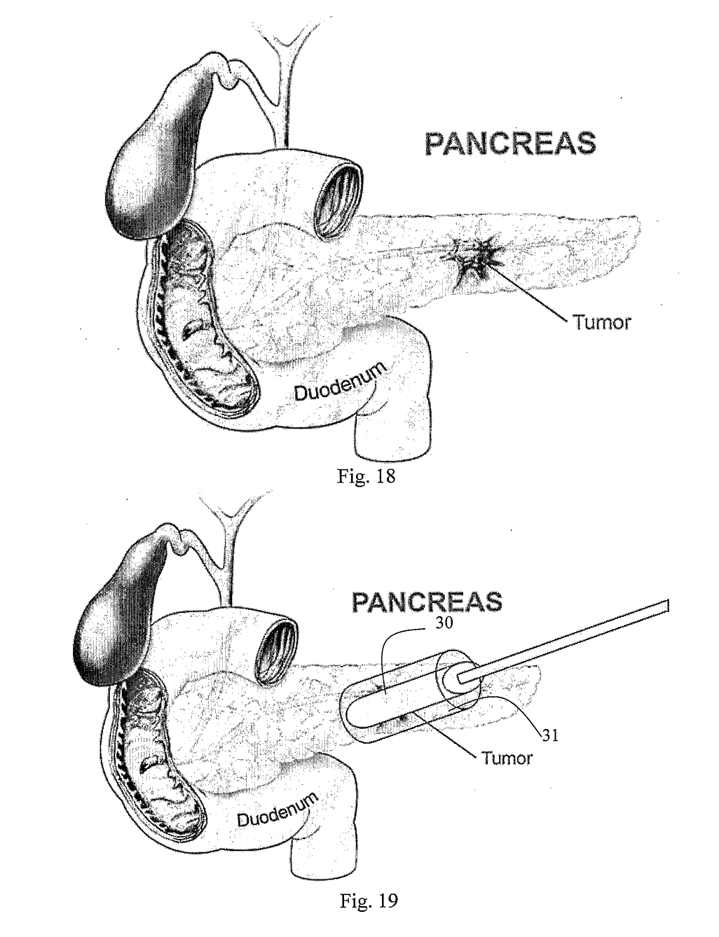

Reference is now made to FIGS. 18-19 which illustrate the above mentioned use. FIG. 18 illustrates the pancreas in which a tumor has developed.

In FIG. 19 one embodiment of the device of the present invention is used. In this embodiment a double coaxial cylinder shaped balloon is used.

The inner cylinder (illustrated as numerical reference 30) is inserted so as to at least partially separate the tumor from the pancreatic duct and to protect the pancreatic duct such that the gel/filler/drug will not obstruct it. Now that the duct is protected and the caner is at least partially located on the outer surface of the inner balloon, the outer cylindrical balloon (illustrated as numerical reference 31) is inflated and compresses the tumor tissues. The outer balloon also enables the insertion of gel containing slow release chemotherapy agent.

Hence according to said embodiment, the restoration system comprises (a) at least two percutaneously insertable coaxial expandable elements adapted to be (i) inserted into a region of interest in the body; and, (ii) expanded in the same to form a scaffold; said expandable element is characterized by a predetermined 3D shape, such that upon expansion of said two elements, a confined volume within the same is created such that the outer element is adapted to confine said volume; and, (b) an injectable filler having a first flowable state, and a second non-flowable set state;

said filler is configured for being introduced, in said first flowable state, into said confined volume of said scaffold following the full expansion of said two expandable elements in said region of interest so as to prevent any leakage of said filler out of said confined volume.; and, following introduction of said filler into said confined volume of said scaffold, said filler is adapted to set into said second non-flowable state.

According to another embodiment, the outer cylinder may release antibiotic treatment for pancreatitis.

According to another embodiment, the device of the present invention is used in aiding vertebroplasty.

The pre-design shape of the balloon can act as temporary posterior vertebral wall allowing filling a vertebral tumor with cement without risk of cement leakage into the epidural space with ensuing neurological damage.

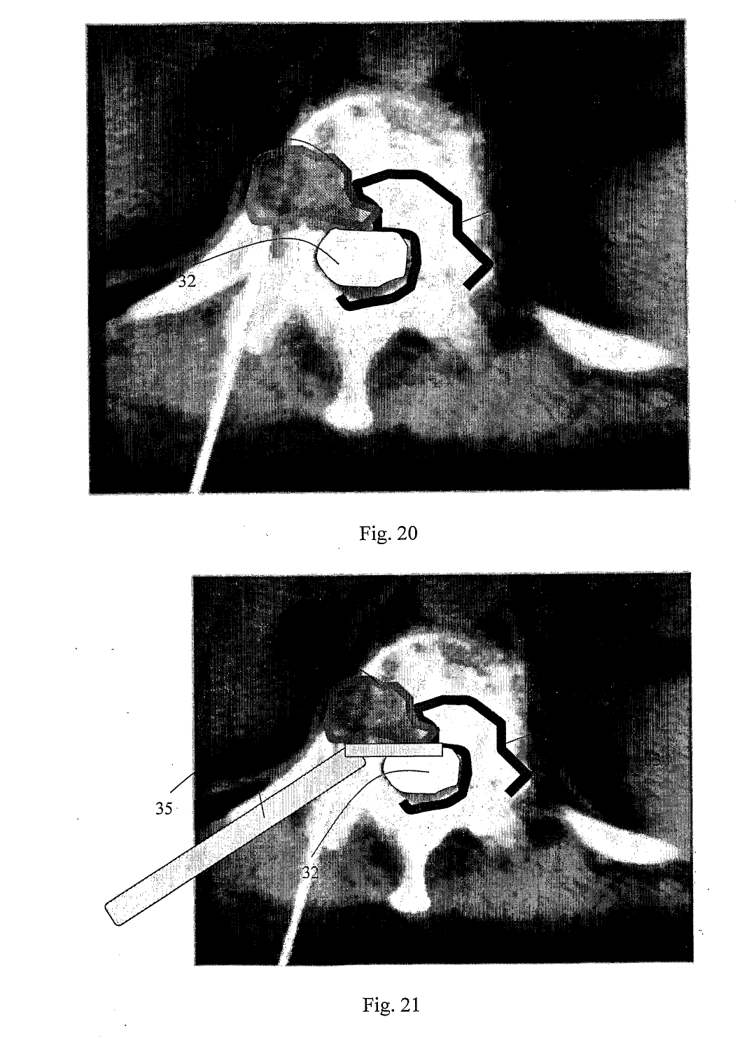

Reference is now made to FIG. 20 which illustrates such an embodiment.

In FIG. 20, numerical reference 32 represents the neural elements; numerical reference represents bone edge; and, numerical reference 34 represents the tumor edge.

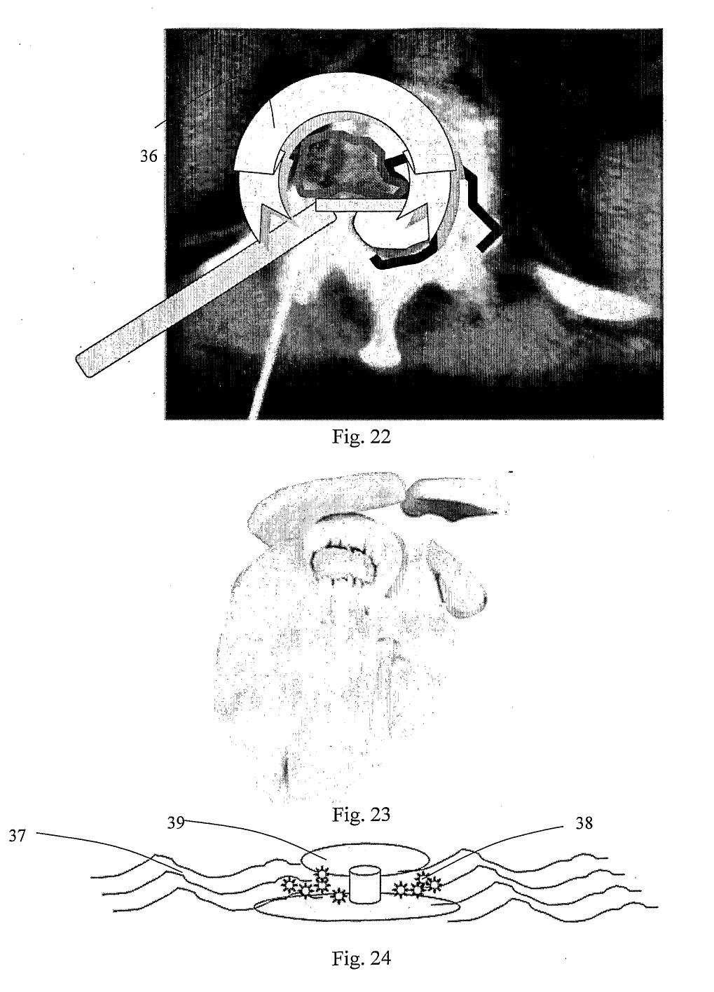

In FIGS. 21-22, the device of the present invention (illustrated as numerical reference 35) is introduced.

The pre-design shape balloon 36 of device 35 acts as a leakage preventing element adapted to prevent the flowable material from reaching the neural elements.

The balloon 36 creates virtual space in the tumor mass to which flowable material is inserted and is restricted to said space.

According to another embodiment, the device of the present invention is used for gluing of tears.

Reference is now made to FIGS. 23-24.

FIG. 23 illustrates the rotator cuff tear. FIG. 24 illustrates the introduction of the device of the present invention into the rotator cuff tear in which numerical reference number 37 illustrates the rotator cuff; numerical reference number 39 illustrates the device; and numerical reference number 38 illustrates the gel.

The special configurations require two balloons which create a closed space within the same; allowing gel injection and gluing of tendon ends.

While the balloon is inflated—its brings into closed contacts the two ends; then, once the two ends are close gel or other gluing material can be introduced which will result in a healing process of the tendon.

According to another embodiment, the inflation of the balloon can lead to instillation of polymer mesh into the tendon to create primary repair.

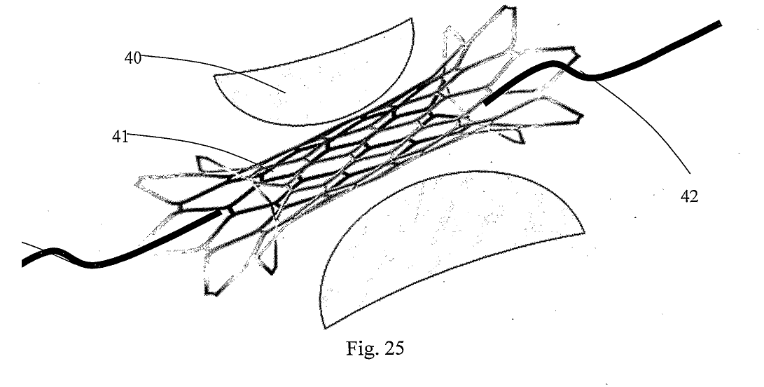

Reference is now made to FIG. 25 which illustrates such an embodiment. In the figure numerical reference number 40 illustrates the balloon, numerical reference 41 illustrates the stent, and numerical reference 42 illustrates the tendon ends.

The balloons 40 are adapted to compress the 41 stent and hence anchoring it into the tendon 42.

While the balloon is inflated—its brings into closed contacts the two ends. This is performed, according to this embodiment, by slowly pressing on the stent, which in turn capture the two ends of the tendon.

Once the two ends are secured and brought into close contact, a curing gel can be released.

While a number of exemplary aspects and embodiments have been discussed above, those of skill in the art will recognize certain modifications, permutations, additions and sub-combinations thereof. It is therefore intended that the following appended claim as and claims hereafter introduced be interpreted to include all such modifications, permutations, additions and sub-combinations as are within their true spirit and scope.

### Examples

Examples are given in order to prove the embodiments claimed in the present invention. The example, which is a clinical test, describes the manner and process of the present invention and set forth the best mode contemplated by the inventors for carrying out the invention, but are not to be construed as limiting the invention.

As described above, the main features of the device are as follows:
(i) the device is percutaneously implantable;
(ii) The device restores disc height;
(iii) The gel is injectable percutaneously;
(iv) The balloon-gel procedure restores mechanical properties of the degenerated disc;
(v) The gel/filler coagulates in situ;
(vi) The gel/filler does not leak provided a balloon elevation device is used
(vii) The gel/filler does not dislodge during at least 3 months follow-up period;
(viii) Osteophyte production is halted after disc injury that should cause disc degeneration;
(ix) Crevice formation within damaged discs is halted;
(x) The gel/filler relieves osteoarthritic pain;
(xi) The gel/filler undergoes slow biodegradation that is completed within 4 months;
(xii) Histologicallly the disc degenerative process is halted and new tissue formation is seen;
(xiii) The gel/filler does not evoke an inflammatory response
(xiv) Biosyntech gel/filler inferiority due to cell toxicity of their gel/filler; and,
(xv) Superiority to hyaluronate in stress conditions that are typical of degenerated discs.

### Example 1

### Restoration of the Disc's Mechanical Properties by the Implantation of the Gel/Filler

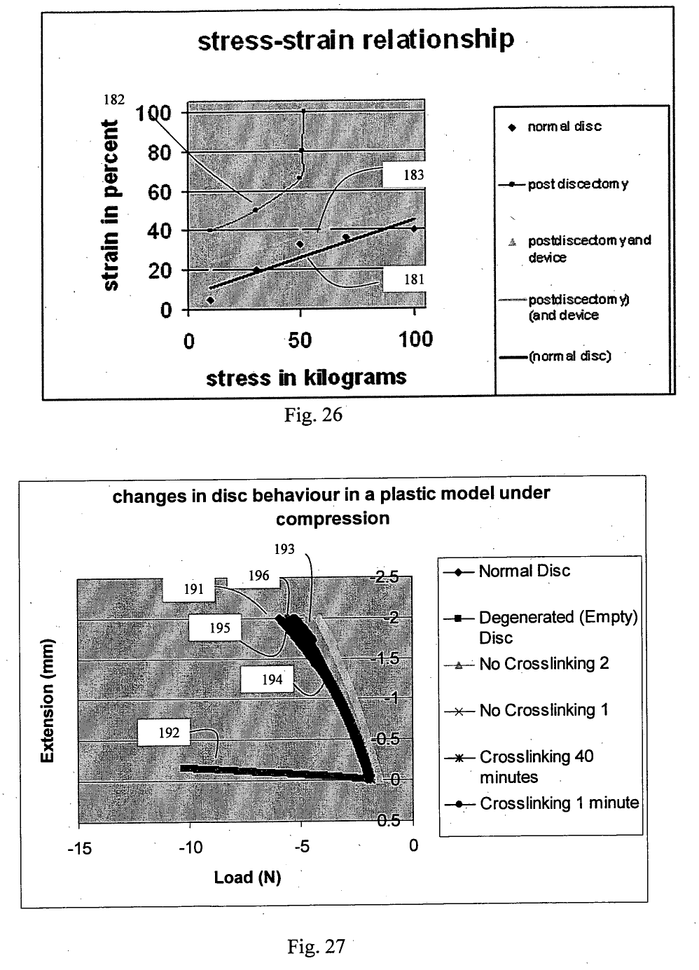

Reference is now made to FIG. 26 which illustrates % strain vs. stress in Kg. As can be seen from the figure a normal disc demonstrates a linear behavior (illustrates as numerical reference 181). Furthermore, the post discectomy mechanical behavior (illustrates as numerical reference 182) illustrates arrival to a yield point is reached after a stress of 50 Kg is exerted.

On the contrary, the post discectomy mechanical behavior when the device of the present invention is used (illustrates as numerical reference 183), demonstrates mechanical behavior which is similar to that of the normal disc (i.e., linear behavior).

Reference is now made to FIG. 26 which illustrates the disc behavior under compression of a normal disc (illustrates as numerical reference 191), a degenerated disc (illustrates as numerical reference 192), a no cross linking—no hyaluronate (illustrates as numerical reference 193), a no cross linking with hyaluronate (illustrates as numerical reference 194), a 40 minutes cross linking with gennipin (illustrates as numerical reference 195), a 1 minute cross linking with gennipin (illustrates as numerical reference 196).

As can be seem from the figure the different examples gave a mechanical result which is similar to the mechanical behavior of the normal disc. In other words, the filler of the present invention restore the mechanical properties close to normal.

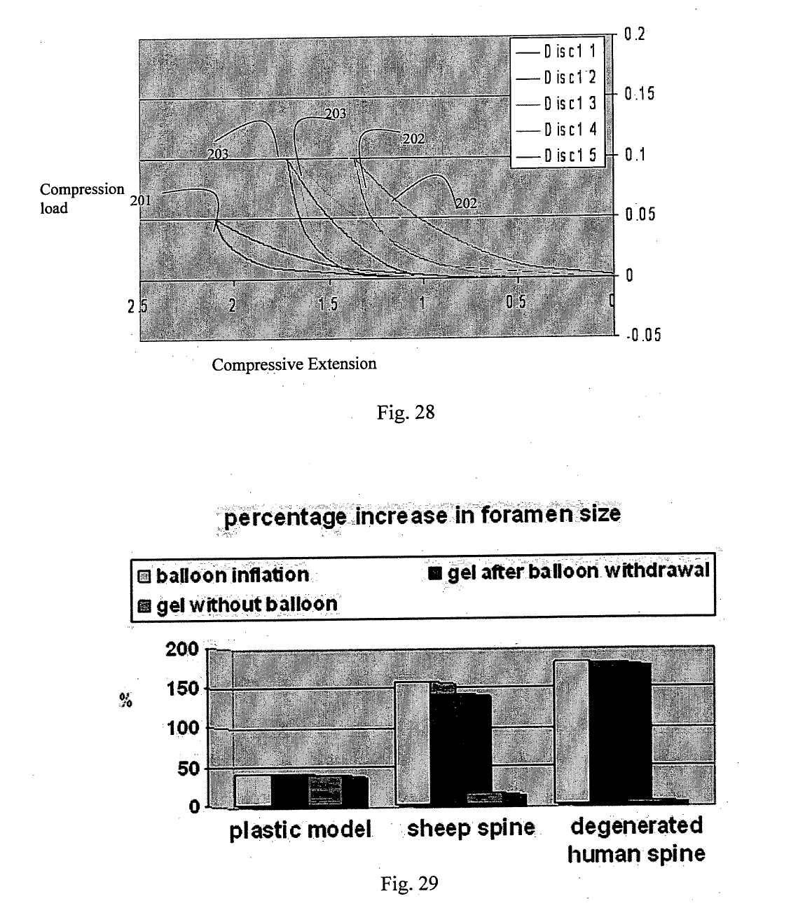

Reference is now made to FIG. 28 which again illustrates the restoration of mechanical properties while using the device of the present invention while the same has been used in sheep discectomy model.

As can be seen from the figure, the degenerated disc (blue, illustrates as numerical reference 201) displaces readily compared with normal (yellow and pink, illustrates as numerical reference 202) while repaired disc (violet turquoise, illustrates as numerical reference 203) behaves similarly to normal discs.

### Example 2

### (%) Increase in Neural Foramen Size

Reference is now made to FIG. 29 which illustrates the increase (%) in neural foramen size in different models plastic model, sheep spine, degenerated human spine.

As can be seen from the figure, while using the balloon, gel and the balloon and gel in plastic model gives the same results, using the balloon and gel combined with the balloon in sheep spine, degenerated human spine provides 6 to 17 times better results.

### Example 3

### Disc Height Increase—Balloon and Gel Vs. Gel Alone

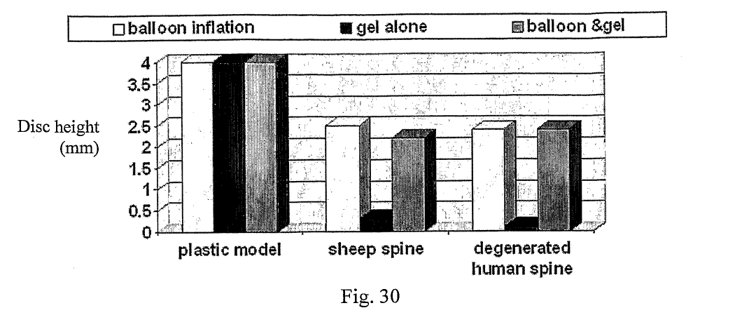

Reference is now made to FIG. 30 which illustrates disc height increase when a balloon and gel/filler are used and when the gel/filler alone is used in different models.

As can be seen from the figure, there is no difference between using the balloon alone, the gel alone or the balloon and gel in the plastic model.

On the other hand performing the same experiment, in sheep's spine using the gel alone provide a relatively small increase in the disc height, while using the balloon and the combined gel and balloon provides 4-5 times more elevation in disc height.

The same is observed in human spine.

The main difference between the balloon alone and the balloon and gel is the fact the balloon maintains disc height disc as long as the balloon is kept inflated. On the other hand the gel injection allows maintenance of disc height constantly without the requirement to keep the balloon inflated.

### Example 4

### Biocompatible and Biodegradable

The gel/filler used in the present invention is both biocompatible and biodegradable. Reference is now made to FIG. 31 which illustrates the palpatory size of the subcutaneous gel/filler used in the present invention.

As can be seen from the figure, the filler of the present invention may degrade within 3-4 months.

### Example 5

### Effect of Gel/Filler of the Present Invention on Intervertebral Disc Mechanical Behavior

In the following examples the model that was used is silicone based with an internal gel/filler.

As it is widely known—the removal of “nucleus pulposus” caused mechanical insufficiency of the disc.

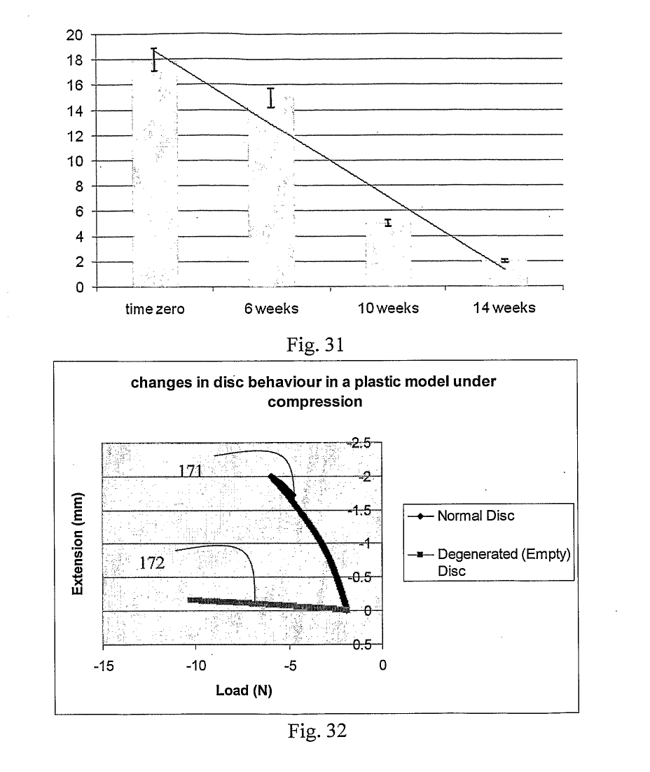

Reference is now made to FIG. 32 which illustrates changes in disc behavior under compression of a normal disc (illustrated as numerical reference 171) and of a degenerated disc (illustrated as numerical reference 172).

### Example 6

### Disc Repair Allows Shock Absorption

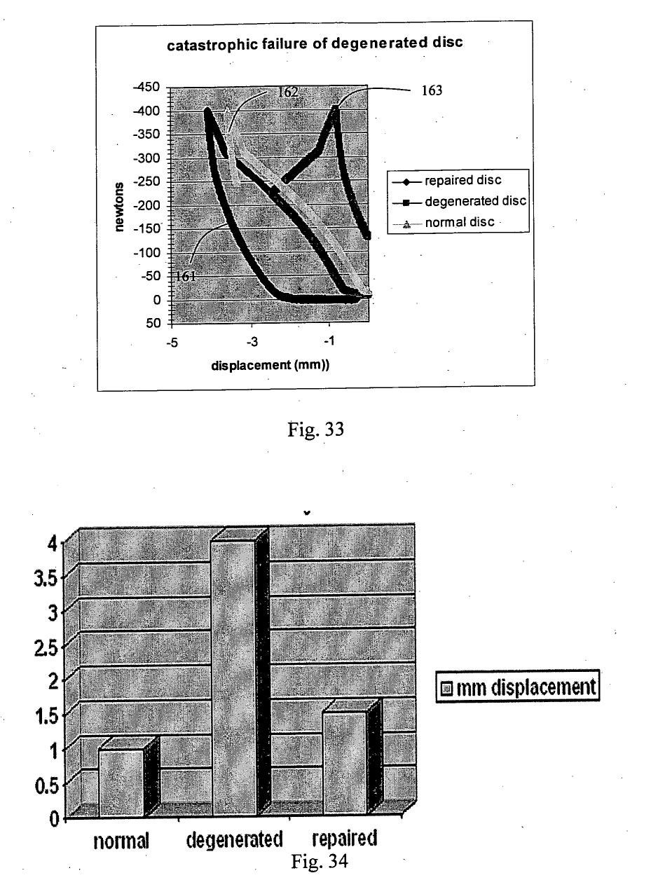

Reference is now made to FIG. 33 which illustrate the ability of the repair disc to shock absorb.

As can be seen from the FIG. 33, while the repaired disc (illustrated as numerical reference 161) behaves similarly to the normal disc (illustrated as numerical reference 162), the degenerated disc (illustrated as numerical reference 163) diverts from the normal behavior.

### Example 7

### Effect on Translational Motion

In this example the parameter that was measured was the amount of translation after 20 kg force application.

Reference is now made to FIG. 34 which illustrate the amount of translation in a normal disc, a degenerated disc and in a repaired disc after 20 Kg force was applied. As can be seen from the figure, the behavior of the normal disc and the repaired once was similar while the behavior of the degenerated one illustrated an amount of translation which is almost 4 times greater than the normal behavior.

### Example 8

### The Degradation of the Gel/Filler

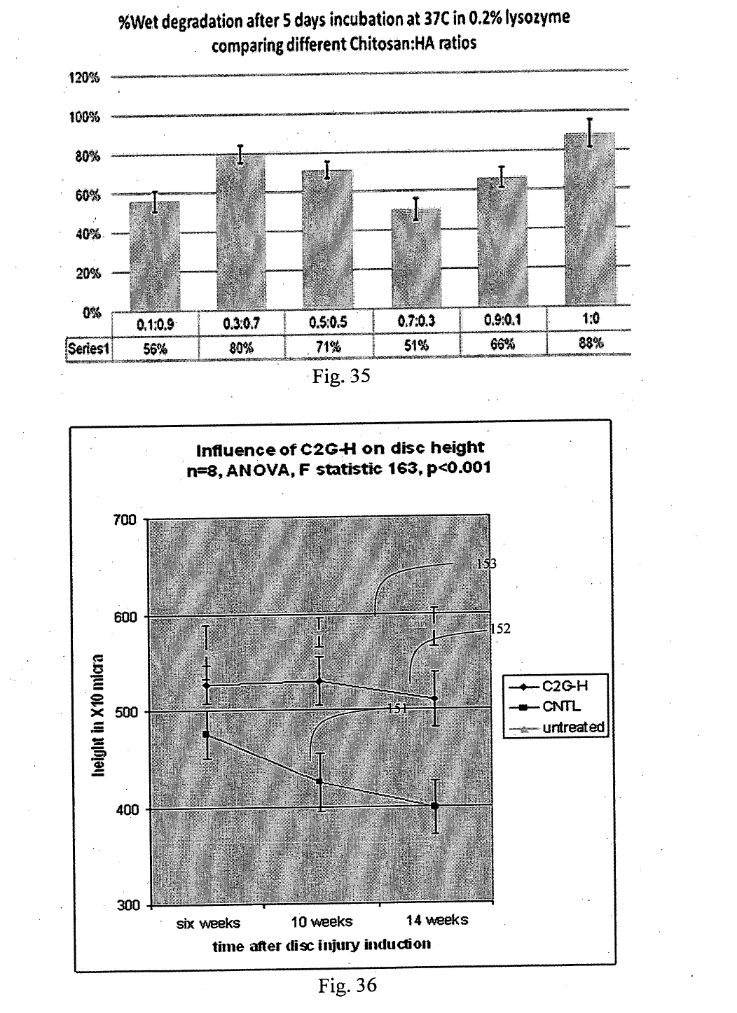

Reference is now made to FIG. 35 which illustrate the % Wet degeneration after 5 days incubation at 37 degrees in 0.2% lysozyme comparing different % of chitosan and Hyaluronate (HA) as the filler.

As can be seen from the figure, a ratio of 0.7:0.3 has the slowest degradation. (n=5).

### Example 9

### Rabbit Model

In the following experimental protocol, 8 rabbits were used. The discs were punctured which causes slow disc degeneration.

In each three discs were examined:
Damaged with chitosan implantation (experimental group)
Damaged without chitosan implantation (control)
Intervening disc not damaged (normal)

It should be noted that the masuda model was used (i.e., Masuda et al recently developed a new rabbit model of mild, reproducible disc degeneration by an anulus needle puncture. This procedure, using 16 to 21-gauge needles with controlled depth, resulted in a slower decrease in disc height than that obtained with the classic stab procedure, The Journal of Bone and Joint Surgery (American). 2006;88:88-94).

It should be further noted that due to the reproducibility and controllability of the degree of degeneration, this new model has been shown to be effective in studying the effect of a variety of molecules, such as growth factors, on the progression of disc degeneration. In studying intervertebral disc degeneration, investigators should be reminded that there is no correlation between disc degeneration and pain.

The results of 3 months follow-up are illustrates in FIG. 36 in which the disc height as a function of time after disc injury induction is illustrated.

As can be seen from the figure, the difference is significant between the control (the rabbits which were damaged without treatment and the treatment), illustrated in the graph as the CNTL (numerical reference 151), with the rabbits treated with device of the present invention, illustrated in the graph as the C2G-H (numerical reference 152) injection at the 10-weeks and 14-weeks time points.

Anova for the 14 weeks sacrifice timepoints is noted on the graph.

As can be further seen from the graph, the height of the treated disc with the device of the present invention is almost the same as the height of the untreated disc's (i.e. the normal discs) height (numerical reference 153). This is in contrast to the control rabbits which were damaged without any treatment.

### Example 10

### Osteophyte Production after Disc Damage in a Masuda Model

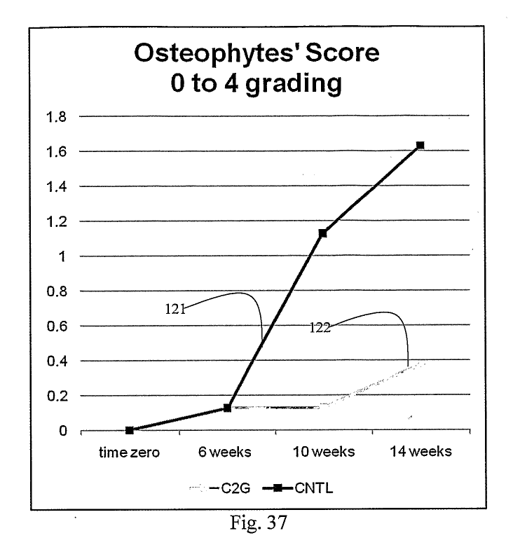

Reference is now made to FIG. 37 which illustrates the Osteophytes' score (0-4 grading) in which 0 means—no osteophytes; 1 means minimal osteophyte; 2 meansosteophyte larger than half disc height; 3 almost touching osteophytes; and, 4 ankylosis.

In the figure, the CNTL (illustrated as numerical reference no. 122) is the control and C2G (illustrated as numerical reference no. 121) refers to the use of the device and gel/filler of the present invention.

As can be seen from the figure, the use of the device of the present invention delays osteophytosis.

1-96. (canceled)

97. A disc restoration system for relieving symptoms of a degenerative disc; wherein said system comprising:
a. a percutaneously insertable expandable element adapted to be (i) inserted in between two adjacent vertebrae of the spinal cord; and, (ii) expanded in the same to form a scaffold; said scaffold provides mechanical support to said two adjacent vertebrae so as to restore said degenerative disc to approximately the dimensions of a normal disc;
said expandable element is characterized by a predetermined 3D shape, such that upon expansion of said element, a confined volume within the same is created;
b. an injectable filler having a first flowable state, and a second non-flowable set state; said filler is configured for being introduced, in said first flowable state, into said confined volume of said scaffold following the full expansion of said expandable element in said disc; and, following introduction of said filler into said confined volume of said scaffold, said filler is adapted to set into said second non-flowable state; such that following setting of said filler into said second state so as to internally support said disc by said set filler; wherein said expandable element is provided with a removable posterior wall supporting means.

98. The disc restoration system of claim 97, wherein said filler is selected from a group consisting of chitosan Fibrin, Hyaluronate, Hydroxyethylcellulose, Biosyntech glycerol phosphate/chitosan, any chitosan hybrid or any combination thereof.

99. The disc restoration system of claim 97, wherein at least one of the following is being held true: (a) said expandable element is injected into the rotator cuff tears in the shoulder so as to limit the movement of said filler in said first flowable state until said filler is set into said second state; (b) said expandable element is configured for being removed from said disc within about 1-60 minutes after insertion of said expandable element, thereby leaving said filler in said set second state supporting said disc in situ; (c) said expandable element is inflatable by a fluid via a lumen; and any combination thereof.

100. The disc restoration system of claim 97, wherein said scaffold includes scaffolding means selected from the group consisting of deployable arms, bulges, struts, swellings, irregular shapes and regular shapes or any combination thereof.

101. The disc restoration system according to claim 97, wherein said scaffold is balloon-like; further wherein at least one of the following is being held true (a) said balloon-like scaffold is provided with means of insertion in an uninflated state between two adjacent vertebrae, further wherein said balloon-like scaffold is inflatable in situ so as to create space between said two adjacent vertebrae so as to provide mechanical support for the same; (b) said balloon-like scaffold is provided with means of filling said space between said vertebrae with bone growth encouraging material; (c) said balloon-like scaffold is provided with means of insertion intrapedicularly into spaces within a vertebral fracture;
further wherein said balloon-like scaffold is inflatable in situ so as to create space within said vertebral fracture; (d) said balloon-like scaffold is provided with means of filling said space within said vertebral fracture with bone growth encouraging material; and any combination thereof.

102. The disc restoration system according to claim 97, wherein said scaffold has interstices that are narrower at a proximal site of injection of said filler into the interior of said raised disc than at sites distal to said site of injection, so as to facilitate gelation and setting of said filler in a proximal to distal direction, such that leakage of said filler is prevented.

103. The disc restoration system according to claim 97, wherein at least one of the following is being held true: (a) said expandable element additionally comprises a plurality of predetermined orifices from which said filler is introduced into said confined volume of said expandable element; (b) said expandable element comprises at least one rigid or semi rigid member; (c) said expandable element is provided with means for sustained drug delivery; (d) said expandable element is provided with means for rapid drug delivery; and any combination thereof.

104. The disc restoration system according to claim 97, wherein said scaffold has a configuration selected from a group consisting of spiral-like configuration, jack-like configuration, serpentine-like configuration and any combination thereof.

105. The disc restoration system according to claim 97, wherein said scaffold is formed from shape memory alloys.

106. The disc restoration system according to claim 97, wherein said filler additionally comprises at least one selected from a group consisting of nano-particles, endogenous or non-endogenous disc-cells and any combination thereof.

107. A method for relieving symptoms of a degenerative disc, comprising steps of;
a. obtaining a disc restoration system, said system comprising;
i. a percutaneously insertable expandable element adapted to be inserted into a degenerative disc of the spinal cord and expanded in said disc to form a scaffold; said scaffold provides mechanical support to said disc so as to restore said disc to approximately the dimensions of a normal disc; said expandable element is characterized by a predetermined 3D shape, such that upon expansion of said element, a confined volume within the same is created;
ii. an injectable filler having a first flowable state, and a second non-flowable set state; wherein said expandable element is provided with a removable posterior wall supporting means;
b. inserting said expandable element into said degenerative disc in a deflated state;
c. fully expanding said element so as to restore said disc to approximately normal disc dimensions;
d. introducing said filler into said confined volume of said expandable element; thereby filling the internal spaces of said restored disc with said filler;
e. waiting for said filler to gelate and set to said second state; and,
f. removing said expandable element.

108. The method according to claim 107, additionally comprising step of providing said expandable element with scaffolding means selected from a group consisting of deployable arms, bulges, struts, swellings, irregular shapes and regular shapes or any combination thereof.

109. The method according to claim 107, additionally comprising step of withdrawing said expandable element from the interior of said disc within about 1-60 minutes following insertion of said expandable element, thereby leaving said filler in said gelated second state supporting said disc in situ.

110. The method according to claim 107, wherein said step of fully expanding said element is performed by a fluid via a lumen.

111. The method according to claim 107, additionally comprising step of selecting said filler from a group consisting of chitosan Fibrin, Hyaluronate, Hydroxyethylcellulose, Biosyntech glycerol phosphate/chitosan or any combination thereof.

112. The method according to claim 107, additionally comprising step of configuring said scaffold to have a balloon-like structure.

113. The method according to claim 107, additionally comprising step of injecting said expandable element into the rotator cuff tears in the shoulder; thereby limiting the movement of said filler in said first flowable state until said filler is set into said second state.

114. The method according to claim 107, wherein said method further comprises steps of:
a. inserting said uninflated balloon-like scaffold between two adjacent vertebrae; and,
b. inflating said balloon-like scaffold in situ so as to create space between said vertebrae; and,
c. filling said space between said vertebrae with bone growth encouraging material.

115. The method according to claim 107, wherein said method further comprises steps of:
a. inserting intrapedicularly said balloon-like scaffold into spaces within a vertebral fracture;
b. inflating said balloon-like scaffold in situ so as to create space within said vertebral fracture; and,
c. filling said spaces within said vertebral fracture with bone growth encouraging material.

116. The method according to claim 107, additionally comprising step of providing said scaffold with interstices that are narrower at the proximal site of injection of said chitosan filler into the interior of said raised disc than at sites distal to said site of injection, thereby facilitating gelation and setting of said chitosan filler in a proximal to distal direction, thereby preventing leakage of said filler .

117. The method according to claim 107, additionally comprising step of configuring said scaffold in a configuration selected from a spiral-like configuration, a serpentine-like configuration, jack-like configuration or any combination thereof.

118. The method according to claim 107, additionally comprising at least one step selected from (a) providing said expandable element with at least one rigid or semi rigid members; (b) forming said scaffold from shape memory alloys; (c) providing said expandable element with a removable posterior wall supporting means; (d) providing said filler with endogenous or non-endogenous disc-cell; and any combination thereof.

119. The method according to claim 107, additionally comprising step of providing said expandable element with means for at least one selected from (a) sustained drug delivery; (b) rapid drug delivery; and any combination thereof.

120. A restoration system; wherein said system comprising:
a. at least one percutaneously insertable expandable element adapted to be (i) inserted into a region of interest in the body; and, (ii) expanded in the same to form a scaffold; said expandable element is characterized by a predetermined 3D shape, such that upon expansion of said element, a confined volume within the same is created;
b. an injectable filler having a first flowable state, and a second non-flowable set state; said filler is configured for being introduced, in said first flowable state, into said confined volume of said scaffold following the full expansion of said expandable element in said region of interest so as to prevent any leakage of said filler out of said confined volume; and, following introduction of said filler into said confined volume of said scaffold, said filler is adapted to set into said second non-flowable state; wherein said expandable element is provided with a removable posterior wall supporting means.

121. The restoration system of claim 120, wherein said expandable element comprises at least two coaxial cylindrically shaped balloons.

122. The restoration system of claim 121, wherein at least one of the following is being held true (a) said region of interest in the body is suspicious to be cancerous; such that said cancer is confined within the inner space between two neighboring balloons; further wherein said filler is a chemotherapy agent released from the outer balloon towards said cancer, such that said restoration system is used for treating said cancer; (b) said region of interest is selected from pancreas, liver and other soft tissue; and any combination thereof.

123. The restoration system of claim 120, wherein said filler is selected from a group consisting of chitosan Fibrin, Hyaluronate, Hydroxyethylcellulose, Biosyntech glycerol phosphate/chitosan or any combination thereof.

124. The restoration system of claim 120, wherein at least one is being held true (a) said expandable element is injected into the rotator cuff tears in the shoulder so as to limit the movement of said filler in said first flowable state until said filler is set into said second state; (b) said expandable element is configured for being removed from said disc within about 1-60 minutes after insertion of said expandable element, thereby leaving said filler in said set second state supporting said disc in situ; (c) said expandable element is inflatable by a fluid via a lumen; and any combination thereof.

125. The restoration system of claim 120, wherein said scaffold includes scaffolding means selected from the group consisting of deployable arms, bulges, struts, swellings, irregular shapes and regular shapes or any combination thereof.

126. The restoration system according to claim 120, wherein said scaffold is balloon-like; further wherein at least one is being held true (a) said balloon-like scaffold is provided with means of insertion in an uninflated state between two adjacent vertebraes, further wherein said balloon-like scaffold is inflatable in situ so as to create space between said two adjacent vertebrae so as to provide mechanical support for the same; (b) said balloon-like scaffold is provided with means of filling said space between said vertebrae with bone growth encouraging material; (c) said balloon-like scaffold is provided with means of insertion intrapedicularly into spaces within a vertebral fracture; further wherein said balloon-like scaffold is inflatable in situ so as to create space within said vertebral fracture; (d) said balloon-like scaffold is provided with means of filling said space within said vertebral fracture with bone growth encouraging material; and any combination thereof.

127. The restoration system according to claim 120, wherein said scaffold has interstices that are narrower at a proximal site of injection of said filler into the interior of said raised disc than at sites distal to said site of injection, so as to facilitate gelation and setting of said filler in a proximal to distal direction, such that leakage of said filler is prevented.

128. The restoration system according to claim 120, wherein said expandable element additionally comprises at least one selected from (a) a plurality of predetermined orifices from which said filler is introduced into said confined volume of said expandable element; (b) comprises at least one rigid or semi rigid member; and any combination thereof.

129. The restoration system according to claim 120, wherein said scaffold has a configuration selected from a group consisting of serpentine-like configuration, spiral-like configuration, jack-like configuration; and any combination thereof.

130. The restoration system according to claim 120, wherein said scaffold is formed from shape memory alloys.

131. The restoration system according to claim 120, wherein at least one of the following is being held true: (a) said expandable element is provided with means for sustained drug delivery; (b) said expandable element is provided with means for rapid drug delivery; and any combination thereof.

132. The restoration system according to claim 120, wherein said filler additionally comprises at least one selected from a group consisting of nano-particles, endogenous or non-endogenous disc-cells; and any combination thereof.

133. The restoration system according to claim 120, wherein said element is adapted to apply pressure on an organ enclosed within said confined volume; further wherein said organ is selected from a group consisting of tendon, Pancreas, liver and other soft tissue.

134. A method for treating a body organ, comprising steps of;
a. obtaining a restoration system, said system comprising;
i. at least one percutaneously insertable expandable element adapted to be (i) inserted into a region of interest in the body; and, (ii) expanded in the same to form a scaffold; said expandable element is characterized by a predetermined 3D shape, such that upon expansion of said element, a confined volume within the same is created;
ii. an injectable filler having a first flowable state, and a second non-flowable set state; wherein said expandable element is provided with a removable posterior wall supporting means;
b. inserting said two expandable element into said region of interest;
c. expanding said inner element so as to circumscribe said body organ;
d. expanding said outer element thereby creating said confined volume;
e. introducing said filler into said confined volume thereby treating said cancer; and,
f. removing said expandable element.

135. The method according to claim 134, additionally comprising step of providing said expandable element with at least two coaxial cylindrically shaped balloons.

136. The method according to claim 135, wherein said region of interest in the body is suspicious to be cancerous; such that said cancer is confined within the inner space between two neighboring balloons; further wherein said filler is a chemotherapy agent released from the outer balloon towards said cancer, such that said restoration system is used for treating said cancer.

137. The method according to claim 134, wherein said organ in said region of interest is suspicious of cancer; further wherein said organ is selected from a group consisting of tendon.

138. The method according to claim 134, additionally comprising step of providing said expandable element with scaffolding means selected from a group consisting of deployable arms, bulges, struts, swellings, irregular shapes and regular shapes or any combination thereof.

139. The method according to claim 134, additionally comprising step of withdrawing said expandable element from the interior of said disc within about 1-60 minutes following insertion of said expandable element, thereby leaving said filler in said gelated second state supporting said disc in situ.

140. The method according to claim 134, wherein said step of fully expanding said element is performed by a fluid via a lumen.

141. The method according to claim 134, additionally comprising step of selecting said filler from a group consisting of chitosan Fibrin, Hyaluronate, Hydroxyethylcellulose, Biosyntech glycerol phosphate/chitosan or any combination thereof.

142. The method according to claim 134, additionally comprising step of configuring said scaffold to have a balloon-like structure.

143. The method according to claim 134, additionally comprising step of injecting said expandable element into the rotator cuff tears in the shoulder; thereby limiting the movement of said filler in said first flowable state until said filler is set into said second state.

144. The method according to claim 134, wherein said method further comprises steps of:
a. inserting said uninflated balloon-like scaffold between two adjacent vertebrae; and,
b. inflating said balloon-like scaffold in situ so as to create space between said vertebrae.

145. The method according to claim 144, further comprising steps of filling said space between said vertebrae with bone growth encouraging material.

146. The method according to claim 134, wherein said method further comprises steps of:
a. inserting intrapedicularly said balloon-like scaffold into spaces within a vertebral fracture;
b. inflating said balloon-like scaffold in situ so as to create space within said vertebral fracture.

147. The method according to claim 146, further comprising step of filling said spaces within said vertebral fracture with bone growth encouraging material.

148. The method according to claim 134, additionally comprising step of providing said scaffold with interstices that are narrower at the proximal site of injection of said chitosan filler into the interior of said raised disc than at sites distal to said site of injection, thereby facilitating gelation and setting of said chitosan filler in a proximal to distal direction, thereby preventing leakage of said filler.

149. The method according to claim 134, additionally comprising step of configuring said scaffold in a configuration selected from a spiral-like configuration, a serpentine-like configuration, a jack-like configuration or any combination thereof.

150. The method according to claim 134, additionally comprising step of providing said expandable element with at least one rigid or semi rigid members.

151. The method according to claim 134, additionally comprising step of forming said scaffold from shape memory alloys.

152. The method according to claim 134, additionally comprising step of providing said expandable element with means adapted for at least one selected from a group consisting of (a) sustained drug delivery; (b) rapid drug delivery; and any combination thereof.

153. The method according to claim 134, additionally comprising step of providing said expandable element with a removable posterior wall supporting means.

154. The method according to claim 134, additionally comprising step of providing said filler with endogenous or non-endogenous disc-cells.
